(19) United States
(12) Patent Application Publication
(10) Pub. No.: US 2015/0168425 A1
(43) Pub. Date: Jun. 18, 2015

(54) METHOD AND AGENTS TO QUANTIFY PROTEINS FROM TISSUES

(71) Applicant: Agada Biosciences, LLC, Halifax (CA)

(72) Inventors: Yetrib Hathout, Silver Spring, MD (US); Kristy Brown, Rockville, MD (US); Eric Hoffman, Kensington, MD (US)

(21) Appl. No.: 14/573,984

(22) Filed: Dec. 17, 2014

Related U.S. Application Data
(60) Provisional application No. 61/917,153, filed on Dec. 17, 2013.

(51) Int. Cl. G01N 33/68

(52) U.S. Cl. CPC G01N 33/6887; G01N 2333/4712

(57) ABSTRACT

As described herein, a robust generalizable method to accurately and sensitively quantitate proteins in tissues and fluids from human patients in a direct manner, not reliant on antibody reagents has been developed. This provides new and robust methods that are useful as biochemical outcome measures in human clinical trials. The methods where mouse tissues fully labeled with stable isotopes are mixed with the human tissue of interest, and the mixture subjected to high throughput proteomics methods are provided. The method detects multiple protein peptides showing 100% conservation between human and mouse, and establishes test and control proteins for quantitation of surrogate biomarkers. This assay method provides a high degree of reliability and sensitivity, as well as generalizability, compared to more traditional antibody-based assays. The specific peptide pairs and methods of quantitation are also described. In an additional embodiment, labeled standards may be derived from labeled human cell culture, such as a myotube culture.

### RELATED APPLICATIONS

This application claims priority to U.S. Provisional Application No. 61/917,153 filed Dec. 17, 2013, the contents of which are incorporated herein in its entirety and for all purposes.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 100175_{—}401_SEQUENCE LISTING.txt. The text file is 1.2 MB, was created on Dec. 16, 2014, and is being submitted electronically via EFS-Web.

### FIELD OF THE INVENTION

The invention generally relates to methods and agents that enable the sensitive and reliable detection and quantification of proteins of interest in human tissues from patients enrolled in clinical trials (surrogate biochemical biomarker). The invention enables and facilitates regulatory review by providing robust measurements of surrogate biochemical biomarkers at accuracies far exceeding previous methods.

### BACKGROUND OF THE INVENTION

Bringing a new drug (new chemical entity; NCE) to market and to the benefit of patients requires regulatory agency approvals, and such approvals require clinical trials. Proving that a NCE shows efficacy often depends on showing improvement of clinical outcome measures, typically related to patient quality of life. However, clinical outcome measures often require clinical trials of long duration, and the endpoints can be subjective and variable. These increase the number of patients needed for a robust clinical trial, their cost, and time required.

An alternative to clinical endpoints in clinical trials are surrogate biochemical biomarkers as endpoints (1). A surrogate biochemical endpoint should have a compelling rationale for being reflective of the underlying disease process, and should be able to be measured using sensitive and reliable assay methods. If robust biomarkers are identified, then consideration of accelerated approval for a drug may be based on the biomarker data alone, without associated clinical endpoint data. One of the more compelling connections between a disease and a biomarker is with regards to inherited disease, where gene mutations lead to loss of the corresponding protein product in a tissue. This inherited inborn error of metabolism initiates the cascade of pathologies that lead to the patient's clinical picture. An increasing number of drug development programs aim to replace the missing protein in patient tissue, thus directly addressing the primary biochemical defect. As the connection between the primary gene/protein deficiency and the disease process is compelling then quantifying the replacement of this same protein in clinical trials is generally viewed as a valid biochemical surrogate endpoint.

For example, dystrophin is a relatively large (427 kDa) multi-domain protein that plays an essential role in muscle fiber integrity and function. It is predominantly expressed in skeletal muscle by other isoforms have been found expressed in heart and brain (15). Dystrophin is a fairly low abundance protein, accounting for approximately 0.002% of the total striated muscle protein (2). Gene mutations resulting in the complete loss of dystrophin protein expression are the cause of Duchenne muscular dystrophy (DMD), the most common and severe form of muscular dystrophy (16). Mutations resulting in the production of a truncated but partially functional dystrophin protein give rise to a milder dystrophinopathy termed Becker muscular dystrophy (BMD). In general, BMD patients have longer lives and less severe clinical presentations than DMD patients. This is mainly due to the presence of the partially functional dystrophin protein in BMD patients (17). But, BMD patients do have a wide variety of phenotypes ranging from mild to severe most probably due to the variable amounts of expressed dystrophin often seen in BMD population (18).

The need to accurately detect and quantitate dystrophin in human skeletal muscle biopsies is becoming crucial as new promising therapies aiming to restore dystrophin expression, especially in DMD patients, are entering phase II/III clinical trials (3-7). Perhaps one of the most promising new generation therapies for DMD is “exon skipping” which has been effective in restoring dystrophin expression and muscle force in both mouse and dog models of DMD (19, 20). The goal of this approach is to “skip” an exon neighboring a deletion mutation during pre-mRNA splicing, thereby restoring the mRNA reading frame, resulting in the production of a shorter length, partially functional Becker-like dystrophin protein. As such, quantification of dystrophin protein is an ideal endpoint measure and clinically meaningful since it is directly linked to the disease.

Most existing methods used to quantify proteins rely on antibodies, and assays association with antibody binding to the target protein. Antibody-based methods, such as immunoblotting, immunostaining, and ELISA methods are inherently indirect methods of assaying proteins, and are typically associated with assay-to-assay variability due to the indirect nature of the tests. Also, robust immunological assays for a target protein require well characterized and highly specific antibody reagents, yet these do not exist for the large majority of target proteins that may be used as surrogate biochemical endpoints in clinical trials. Thus, there is a need to directly measure proteins in patient tissue, in order to develop robust biochemical endpoints for clinical trials.

### SUMMARY OF THE INVENTION

As described herein, a robust generalizable method to accurately and sensitively quantitate protein of interest in tissues and body fluids from human patients in a direct manner, not reliant on antibody reagents has been developed. This provides new and robust methods that are useful as biochemical outcome measures in human clinical trials. Methods where mouse tissues, or human cells in culture, fully labeled with stable isotopes are mixed with the human tissue of interest, and the mixture subjected to precision mass spectrometry proteomics methods are provided. The method detects multiple protein peptides showing 100% conservation between human and mouse (or between human and human cell culture), and establishes test and control proteins for quantitation of surrogate biomarkers. This assay method provides a high degree of reliability and sensitivity, as well as generalizability, compared to more traditional antibody-based assays. The specific peptide pairs and methods of quantitation are also described.

According to an aspect of the present invention, there is provided a method for identifying candidate peptide markers for a protein from a biological sample of interest. The method comprising the steps of: extracting proteins from the biological sample of interest and a corresponding biological sample from an animal that has been labeled with one or more stable isotopic amino acids; mixing a quantity of protein from the biological sample of interest with a quantity of protein from the corresponding biological sample from the labeled animal to form a spiked sample; separating the spiked sample based on a physical property; digesting the proteins of interest using a protease; subjecting the digested samples to mass spectrometry analysis; and identifying unique candidate peptide markers that correspond to a protein.

In another embodiment of the present invention, the biological sample that has been labeled with one or more stable isotopic amino acids may be obtained from human cell culture, such as a human muscle cell line (myotubes) culture.

According to another aspect of the present invention, there is provided a method for quantifying expression of a biomarker from a biological sample obtained from a subject with a disease or disorder. The method comprising the steps of: extracting proteins from the biological sample of interest; mixing a known quantity of protein from the biological sample of interest with a known quantity of protein a the corresponding biological sample from a labeled animal or a labeled synthetic peptide corresponding to a protein of interest to form a spiked sample; separating the spiked sample based on a physical property; digesting the proteins of interest using a protease; subjecting the digested samples to mass spectrometry analysis; determining intensities of unique candidate peptide markers that correspond to a proteins of interest; and quantifying the amount of the protein of interest in the sample based on the relative intensity of the unique candidate peptide markers. In another embodiment, the labeled sample may be obtained from human cell culture, such as a human muscle cell line (myotubes) culture.

In one embodiment, the step of quantifying comprises comparing peak areas and area ratios of one or more unique candidate peptide markers from the biological sample of interest to the peak areas and area ratios of one or more labeled peptide markers.

In another embodiment, the step of quantifying comprises comparing the relative intensities to a standard curve generated by creating a series of dilutions of a biological sample from a normal subject with a biological sample from a subject with a disease or disorder, wherein the amount of total protein in the mixed sample remains constant; mixing a known quantity of protein from the mixed sample with a known quantity of protein a corresponding biological sample from a labeled animal or a labeled synthetic peptide corresponding to a protein of interest to form a spiked sample; separating the spiked sample based on a physical property; digesting the proteins of interest using a protease; subjecting the digested samples to mass spectrometry analysis; and generating a titration curve based on the intensities obtained for each sample. In another embodiment, the labeled sample may be obtained from human cell culture, such as a human muscle cell line (myotubes) culture.

According to a further aspect of the invention, there is provided a method for quantifying dystrophin expression from a biological sample obtained from a subject with Duchenne's Muscular Dystrophy. The method comprising the steps of: extracting proteins from the biological sample of interest; mixing a known quantity of total protein from the biological sample of interest with a known quantity of total protein a the corresponding biological sample from a labeled animal, a labeled human cell culture such as a human muscle cell line (i.e. myotubes) culture, or a labeled synthetic peptide corresponding to dystrophin to form a spiked sample; separating the spiked sample based on a physical property; digesting the proteins of interest using a protease; subjecting the digested samples to mass spectrometry analysis; and quantifying the amount of the protein of interest in the sample based on the relative intensity of the unique candidate peptide markers.

In one embodiment, the step of quantifying comprises comparing peak areas and area ratios of one or more unique candidate peptide markers from the biological sample of interest to the peak areas and area ratios of one or more labeled peptide markers.

In another embodiment, the step of quantifying comprises comparing the relative intensities to a standard curve generated by creating a series of dilutions of a biological sample from a normal subject with a biological sample from a subject with a disease or disorder, wherein the amount of total protein in the mixed sample remains constant; mixing a known quantity of protein from the mixed sample with a known quantity of protein a corresponding biological sample from a labeled animal, a labeled human cell culture such as a human muscle cell line (i.e. myotubes) culture, or a labeled synthetic peptide corresponding to a protein of interest to form a spiked sample; separating the spiked sample based on a physical property; digesting the proteins of interest using a protease; subjecting the digested samples to mass spectrometry analysis; and generating a titration curve based on the intensities obtained for each sample.

In a further embodiment, the subject is being treated with a pharmaceutical composition.

In a yet further embodiment, the one or more peptides are selected from QAPIGGDFPAVQK (SEQ ID NO: 6463), VLSQIDVAQK (SEQ ID NO: 6445), IFLTEQPLEGLEK (SEQ ID NO: 6441), TLNATGEEIIQQSSK (SEQ ID NO: 940), and VHALNNVNK (SEQ ID NO: 6465).

In a still further embodiment, the proteins are extracted using a buffer comprising 10% sodium-dodecyl sulfate (SDS).

In an embodiment, the known quantity of total protein corresponding to the biological sample from a labeled animal, or from a labeled human cell culture (i.e. a muscle cell culture, or a myotube cell culture) is half the known quantity of total protein from the biological sample of interest.

In another embodiment, the known quantity of total protein corresponding to the biological sample of interest is 50 μg and the known quantity of total protein the corresponding to the biological sample from a labeled animal or from a labeled human cell culture (i.e. a muscle cell culture, or a myotube cell culture) is 25 μg.

In a further embodiment, the protease is trypsin.

In a yet further embodiment, the spiked sample is separated based on size, charge, shape or a combination thereof using electrophoresis.

In a still further embodiment, the spiked sample is separated using gel electrophoresis.

In one embodiment, the method further comprising the step of excising a portion of the gel containing the proteins of interest prior to the digesting step.

### BRIEF DESCRIPTION OF THE DRAWINGS

These and other features, aspects and advantages of the present invention will become better understood with regard to the following description and accompanying drawings wherein:

FIG. 1 is a flow chart showing the experimental design used to quantitate dystrophin protein in human skeletal muscle. Left panel shows the post-digestion spike-in approach where 50 μg total muscle protein extract from human bisopsie were fractionated on SDS-PAGE using 3-8% Tris-Acetate gel, which is suitable for high molecular mass proteins. Then the area corresponding to dystrophin migration level (dashed blue lines) was serially sliced, in-gel digested by trypsin and spiked with stable isotope labeled standard peptides standards for subsequent LC-MS/MS analysis and peptide quantitation. In the Pre-digestion spike-in approach (right panel), 50 μg total muscle protein extract was spiked with 25 μg of ¹³C₆-Lysine labeled SILAC mouse muscle protein extract. The mixture was then fractionated by SDS-PAGE and the area corresponding to dystrophin migration was excised, in-gel digested and the resulting peptides were analyzed by LC-MS/MS for identification and quantitation;

FIG. 2 shows representative mass spectra of spiked-in labeled standard peptides and corresponding unlabeled endogenous peptide using a post-digestion spike-in approach (A) Mass spectra of labeled and unlabeled peptide pairs detected after spiking in-gel digested dystrophin with varying amounts of ¹³C₆, ¹⁵N₂ Lysine labeled standard peptide [IFLTEQPLEGLEK] (SEQ ID NO: 6441). (B) Shows mass spectra of labeled and unlabeled peptide pairs detected after spiking in-gel digested dystrophin with varying amounts of 13C6, 15N2 Lysine labeled standard peptide [LLDLLEGLTGQK] (SEQ ID NO: 937), (C) Shows mass spectra of labeled and unlabeled peptide pairs detected after spiking in-gel digested dystrophin with varying amounts of 13C6, 15N4 Lysine labeled standard [LLVEELPLR] (SEQ ID NO: 6453). The concentration of each spike-in standard peptide, starting for the top to the low panels, was as follows (160 nM, 80 nM, 40 nM and 14 nM). Samples were analyzed on the LTQ-Orbitrap XL at 30,000 resolution and mass error better than 10 ppm. All ions were detected as doubly protonated species with the correct m/z values;

FIG. 3 represents mass spectrometry data obtained for triplicate experiments using the same extract from a muscle biopsy obtained from a healthy donor. Three aliquots of 50 μg total muscle protein extract were separated by SDS-PAGE as described in Methods. The migration area corresponding to dystrophin was excised, in-gel digested and spiked with optimal concentration (30 nM) of stable isotope labeled standard peptides. The left panel shows MS spectra of unlabeled peptide [IFLTEQPLEGLEK] (SEQ ID NO: 6441) generated from endogenous dystrophin and the spike-in stable isotope labeled standard peptide [IFLTEQPLEGLEK*] (SEQ ID NO: 6441). Both peptides were detected as doubly charged ions at their corresponding m/z value of 758.92 and 762.92, respectively. The right panel shows extracted ion chromatograms used for ratio measurement of unlabeled endogenous dystrophin peptide (blue trace) to the stable isotope labeled standard peptide (red trace);

FIG. 4 is a graphical representation showing inter sample reproducibility of the post-digestion spike-in approach. Three samples containing varying amount of dystrophin obtained by mixing muscle extract from a DMD and age matched healthy donor at ratio of 25/75, 50/50 and 75/25 respectively were evaluated for dystrophin quantitation using the post-digestion spike-in approach with 30 nM of stable isotope standard peptides. Histograms represent intensity ratios of endogenous dystrophin peptides and filamin c peptide to their corresponding stable isotope labeled standard peptide. P1, P2 and P3 corresponds to dystrophin peptides [LLDLLEGLTGQK] (SEQ ID NO: 937), [LLVEELPLR] (SEQ ID NO: 6453), [IFLTEQPLEGLEK] (SEQ ID NO: 6441) respectively and P4 correspond to filamin c peptide [VYNVTYTVK] (SEQ ID NO: 2830) that was used as an internal control. It is clearly seen that some ratios did not decrease with decreasing amounts of dystrophin, especially for the peptide Pl, showing high variability for this approach;

FIG. 5 is a graphical representation showing a titration curve of dystrophin protein and other muscle specific proteins using predigestion spike-in approach. Dystrophin protein, as well as myomesin and filamin c, were quantitated in different combination mixtures of muscle extracts from DMD and age matched healthy donors at ratios leading to 0, 5, 10, 25, 50, 75, 90, 95 and 100% of dystrophin relative to normal. Each final mixture contained 50 μg of total muscle protein and was spiked with 25 μg of ¹³C₆-Lysine labeled SILAC mouse muscle extract. Gel bands encompassing dystrophin protein and other muscle specific proteins were excised and processed for mass spectrometry analysis as described in Methods. Transition ion intensities obtained at the MS/MS level for the targeted labeled and unlabeled peptide pairs generated from the spike-in standard and endogenous human dystrophin were used to determine the relative amount of dystrophin and other muscle proteins (myomesin and filamin c). Standard deviation at each data point represents average ratios obtained from labeled and unlabeled peptide pairs: 5 pairs for dystrophin protein (QAPIGGDFPAVQK (SEQ ID NO: 6463), VLSQIDVAQK (SEQ ID NO: 6445), IFLTEQPLEGLEK (SEQ ID NO: 6441), TLNATGEEIIQQSSK (SEQ ID NO: 940), VHALNNVNK (SEQ ID NO: 6465)); 3 pairs for filamin c (SPFVVNVAPPLDLSK (SEQ ID NO: 2850), EVGEHVVSVRK (SEQ ID NO: 6501), HIGISFTPK (SEQ ID NO: 2822)) and two pairs for myomesin (VSEPVAALDPAEK (SEQ ID NO: 2270), VLGGLPDWTIQEGK (SEQ ID NO: 6502)). These peptides have 100% sequence homology between human and mouse 100% sequence homology between human and mouse and were used for ratio measurements. As expected, dystrophin protein increased with increasing relative amount of normal muscle extract while myomesin and filamin c remained unchanged; and

FIG. 6 is a graphical representation showing calculated absolute amount of dystrophin in muscle biopsies obtained from BMD and age matched healthy controls. Total protein extract (50 μg) from each biopsy was spiked with 25 μg of C₆-Lysine labeled SILAC mouse muscle extract and processed using the pre-digestion spike-in approach (see FIG. 1 and Methods). Intensity ratios of labeled and unlabeled peptide pairs were used to determine the absolute amount of dystrophin in each muscle extract.

### DETAILED DESCRIPTION OF THE INVENTION

Described herein are methods for identifying candidate peptide markers for a protein from a biological sample of interest, as well as for quantifying a protein from a biological sample of interest. In some embodiments, methods for quantitating specific proteins in a biological sample of interest using labeled peptides and mass spectrometry analysis are provided. It will be appreciated that the embodiments described herein are for illustrative purposes, and not intended to be limiting in any way.

Without wishing to be limiting in any way, it is envisioned that some or all of the following steps may be performed when carrying out an embodiment of a method for identifying candidate peptide markers for a protein from a biological sample of interest:
extracting proteins from a biological sample of interest,
obtaining a labeled protein and/or peptide sample with peptide fragments and/or protein corresponding to one or more candidate peptide markers for a protein,
mixing a quantity of protein from the biological sample of interest with a quantity of the labeled protein and/or peptide sample to produce a spiked sample,
separating at least a portion of the spiked sample based on a physical property using, for example, gel electrophoresis,
obtaining a separated sample comprising at least the one or more protein(s) of interest,
digesting the separated sample using, for example, a protease,
analyzing the digested sample using mass spectrometry analysis, and
identifying unique candidate peptide markers that correspond to a protein.

In certain embodiments, it is envisioned that some or all of the following steps may be performed when carrying out an embodiment of a method for identifying candidate peptide markers for a protein from a biological sample of interest:
extracting proteins from a biological sample of interest,
obtaining a labeled protein and/or peptide sample with peptide fragments and/or protein corresponding to one or more candidate peptide markers for a protein,
separating at least a portion of the extracted protein sample based on a physical property using, for example, gel electrophoresis,
digesting the separated sample using, for example, a protease, using, for example, in-gel digestion,
obtaining a separated sample comprising at least the one or more protein(s) of interest,
mixing a quantity of the separated sample with a quantity of the labeled protein and/or peptide sample to produce a spiked sample,
analyzing the spiked sample using mass spectrometry analysis, and
identifying unique candidate peptide markers that correspond to a protein.

Without wishing to be limiting in any way, it is envisioned that some or all of the following steps may be performed when carrying out an embodiment of a method for identifying candidate peptide markers for a protein from a biological sample of interest:
extracting proteins from a biological sample of interest,
obtaining a labeled protein and/or peptide sample with peptide fragments and/or protein corresponding to one or more candidate peptide markers for a protein,
mixing a quantity of protein from the biological sample of interest with a quantity of the labeled protein and/or peptide sample to produce a spiked sample,
separating at least a portion of the spiked sample based on a physical property using, for example, gel electrophoresis,
obtaining a separated sample comprising at least the one or more protein(s) of interest,
digesting the separated sample using, for example, a protease,
analyzing the digested sample using mass spectrometry analysis, and
identifying unique candidate peptide markers that correspond to a protein.

In certain embodiments, it is envisioned that some or all of the following steps may be performed when carrying out an embodiment of a method for identifying candidate peptide markers for a protein from a biological sample of interest:
extracting proteins from a biological sample of interest,
obtaining a labeled protein and/or peptide sample with peptide fragments and/or protein corresponding to one or more candidate peptide markers for a protein,
mixing a quantity of protein from the biological sample of interest with a quantity of the labeled protein and/or peptide sample to produce a spiked sample,
separating at least a portion of the spiked sample based on a physical property using, for example, gel electrophoresis,
obtaining a separated sample comprising at least the one or more protein(s) of interest,
digesting the separated sample using, for example, a protease,
analyzing the digested sample using mass spectrometry analysis, and
identifying unique candidate peptide markers that correspond to a protein.

In certain embodiments, the labeled protein and/or peptide sample with peptide fragments and/or protein corresponding to one or more candidate peptide markers for a protein may comprise at least some amino acid sequences with 100% identity to at least a portion of the protein of interest from the biological cample.

In a further embodiment, the biological sample may be any biological sample of interest. By way of example, the biological sample may be a biopsy or other tissue sample. The biological sample may be from a muscle tissue, a brain tissue, a tissue biospsied in clinical trials, blood, serum, plasma, urine, cerebrospinal fluid or other biological sample or biological fluid of interest.

In another embodiment, the biological sample may be spiked with a quantity of labeled protein. The labeled protein, may be labeled with ¹³C6-Lysine, ¹³C6-¹⁵N2-Lysine, ¹³C6-¹⁵N4-Arginine, combinations thereof, or using other appropriate protein labeling techniques (e.g. ¹⁵N labeling or any other amino acid) as will be known to those of skill in the art.

In a further embodiment, at least a portion of the labeled protein may be synthetically prepared using any appropriate method as will be known to a person skilled in the art.

In yet another embodiment, at least a portion of the labeled protein may be derived from a labeled animal. In a further embodiment, the animal may be a mouse.

In yet another embodiment, at least a portion of the labeled protein may be derived from a human cell culture, such as a human muscle cell line (i.e. myotubes) culture.

In certain embodiments, a labeled animal may be a custom “heavy” mouse colony, termed SILAM or SILAC Mouse (stable isotopic labeling by amino acid in a mouse/mammal). In an embodiment, the SILAM or SILAC Mouse may be generated by feeding mice a custom diet containing ¹³C6-Lysine at 1%. In one embodiment, a pregnant dam may be fed with ‘heavy’ diet until a litter (F1 generation) is obtained and continued through weaning of the pups. After weaning, the pups may be continued on ‘heavy’ diet through breeding and generation of a F2 litter. Label incorporation may be monitored for specific organs and specific proteins by mass spectrometry. In some non-limiting embodiments, labeling efficiency may be better than 96% by F2 generation, including skeletal muscle. Other metabolic labeling method of mouse include feeding with ¹⁵N labeled Spirulina where all the nitrogen attoms in a protein are replaced with heavy nitrogen atoms “¹⁵N”

In yet another embodiment, the biological sample may be extracted protein from a biopsied tissue. Extraction may be performed using any appropriate method as will be known to one of skill in the art. In a non-limiting embodiment, the protein may be extracted using RIPA buffer (ThermoScientific) or a modified Laemmli sample buffer containing 10% SDS (10% SDS, 75 mM Tris-HCl, pH 6.8, 10 mM EDTA, 20% glycerol, 50 mM DTT). In some embodiments, the SDS-containing buffer may be preferable.

In certain embodiments, gel electrophorsis, or other appropriate protein separationtechniques known to those skilled in the art such as capillary electrophoresis, HPLC, FPLC, or other methods may be used to separate a biological sample based on weight, size, shape, charge, or other propery.

In some embodiments, the protein from the biological sample may be digested or fragmented using any appropriate technique as will be known to those skilled in the art. By way of example, a trypsin digestion, which may be an in-gel trypsin digestion, may be performed to digest protein in the biological sample.

In a further embodiment, the biological sample may be spiked with labeled protein/peptide post-digestion. In an alternative embodiment, the biological sample may be spiked with labeled protein/peptide pre-digestion. In a further embodiment, the biological sample may be spiked with a known quantity of labeled protein and/or peptide.

In certain embodiments of a method provided herein, the biological sample spiked with labeled protein and/or peptide may be analyzed by mass spectrometry. In some embodiments, mass spectrometry may be used to identify peptide fragments, and corresponding labeled peptide fragments, from a protein of interest. In some embodiments, the identified labeled and unlabeled peptide pairs may be selected as candidate peptide markers for a protein of interest. In additional emobodiments, the labeled and unlabeled peptide pairs detected using mass spectrometry may be used to quantitate one or more proteins in a biological sample.

A wide variety of mass spectrometry (MS) techniques are known in the art, and the extracted samples may be analyzed using any suitable method known in the art. For example, and without wishing to be limiting in any manner, extracts of biological samples are amenable to analysis on essentially any mass spectrometry platform, either by direct injection or following chromatographic separation. Chromatographic separation may involve, by way of example, reverse phase (for example, C18) chromatography, or any other appropriate technique as will be known to those of skill in the art. Typical mass spectrometers are comprised of a source which ionizes molecules within the sample, and a detector for detecting the ionized molecules or fragments of molecules. Non-limiting examples of common sources include electron impact, electrospray ionization (ESI), atmospheric pressure chemical ionization (APCI), atmospheric pressure photo ionization (APPI), matrix assisted laser desorption ionization (MALDI), surface enhanced laser desorption ionization (SELDI), and derivations thereof. Common mass separation and detection systems can include quadrupole, quadrupole ion trap, linear ion trap, time-of-flight (TOF), magnetic sector, ion cyclotron (FTMS), Orbitrap, Q Exactive, and derivations and combinations thereof. In some non-limiting embodiments, MS analysis may be performed using one or both of LTQ-Orbitrap-XL and/or Q Exactive analysis.

In an embodiment, analysis of mass spectrometry data, for example to identify peptide fragments from a protein of interest, may be performed. Analysis may be assissted using, for example, computer software, or any other appropriate technique as will be known to one skilled in the art. Examples of analysis software may include Sequest in the Biowords Browser software (Thermo), Proteome Discoverer 1.3 (ThermoFisher Scientific), Skyline Targeted Proteomics (developed by MacCoss lab/https://skyline.gs.washington.edu/labkey/project/home/software/Skyline/begin.view) and Pinpoint software. MS/MS techniques, or tandem mass spectrometry, which may involve a fragmentation step, may be used during MS sample analysis, and may provide additional peptide fragments and/or data useful for the methods provided herein.

Without wishing to be bound by theory, a protein of interest may be quantified according to an embodiment of a method described herein by comparing the signal intensity of one or more peptide fragments of a protein of interest from a biological sample to corresponding labeled peptide fragments, which may be present in the sample in known amounts. In some non-limiting embodiments, a titration or standard curve may be generated to assist in quantification and to determine limits of detection.

In some embodiments, the protein of interest from the biological sample may be a protein linked to a particular disease. For example, the protein of interest to be quantified may be dystrophin, and the associated disease may be DMD. As will be recognized by those skilled in the art, any of a wide variety of proteins may be quantitated using methods as provided herein, many of which may be linked to a disease or other physiological condition. In some embodiments, a method as provided herein may be used to determine changes in protein levels as a result of disease progression, changes in protein levels as a result of treatment with a drug, or changes in protein levels as a result of other changes affecting a patient.

Those skilled in the art will recognize that a variety of mass spectrometric analysis techniques and instruments may be used to carry out embodiments of the methods provided herein. The examples provided herein are for illustrative purposes, and are not intended to be limiting in any way.

The following examples are provided for the purposes of illustration, and may demonstrate embodiments of at least some of the methods provided herein.

### EXAMPLE 1

### Materials and Methods

### Muscle Biopsies

All human muscle biopsies used in this study were previously banked in a laboratory in accordance with an Institutional Review Board approved protocol at Children's National Medical Center (CNMC) and after obtaining patients written informed consent. Only excess specimens after diagnosis were used. Diagnosis was previously performed at the Center for Genetic Medicine at Children's National Medical Center. Banked specimens were deidentified and only age, gender, diagnosis and phenotype were reported. Total protein extracts of muscle biopsies from healthy subjects were obtained from Dr. David Rowlands at Massey University, Wellington, New Zealand.

### Custom Peptides

Custom dystrophin peptides LLDLLEGLTGQK (SEQ ID NO: 937), LLVEELPLR (SEQ ID NO: 6453), IFLTEQPLEGLEK (SEQ ID NO: 6441) and filamin c peptide VYNVTYTVK (SEQ ID NO: 2830) with 13C6-15N2-Lysine and 13C6-15N4-Arginine were purchased from New England Peptide (Gardner, MA). Peptides were suspended in 50% molecular grade acetonitrile containing 0.1% TFA and spiked into samples prior to mass spectrometric analyses.

### SILAM/SILAC Mouse

Custom “heavy” C57BL6 mice, termed SILAM or SILAC Mouse (stable isotopic labeling by amino acid in a mouse/mammal) were generated in-houseand handled according to Institutional Animal Care and Use Committee guidelines at the Children's National Medical Center. Mice were fed a custom diet containing 13C6-Lysine (Cambridge Isotope Laboratories, Andover, Mass.) at 1%, adhering to laboratory mouse nutritional standards. A pregnant dam was fed with ‘heavy’ diet until a litter (F1 generation) was obtained and continued through weaning of the pups. After weaning, the pups were continued on ‘heavy’ diet through breeding and generation of a F2 litter. Label incorporation was monitored for specific organs and specific proteins by mass spectrometry. Labeling efficiency was better than 96% by F2 generation, including skeletal muscle [8]. This labeling efficiency was similar to labeling efficiencies obtained by others [9]. SILAC labeled skeletal muscles were harvested, flash frozen in liquid nitrogen chilled isopentane and stored at −80° C. until analysis as previously described [8].

### Muscle Protein Preparation

Approximately 50 serial sections were prepared from each of the human muscle biopsies and SILAC mouse gastrocnemius muscle using a cryostat set at 10 μm thickness. Muscle sections were directly placed into a microcentrifuge tube on dry ice. Total protein extraction from the muscle samples was performed using a handheld homogenizer either in a RIPA buffer (ThermoScientific) or a modified Laemmli sample buffer containing 10% SDS (10% SDS, 75 mM Tris-HCl, pH 6.8, 10 mM EDTA, 20% glycerol, 50 mM DTT) [10].Protein concentration was determined using a BCA assay (Pierce, Thermo Scientific).

Dystrophin Quantitation using Mass Spectrometry with Stable Isotope Spike-In Strategy

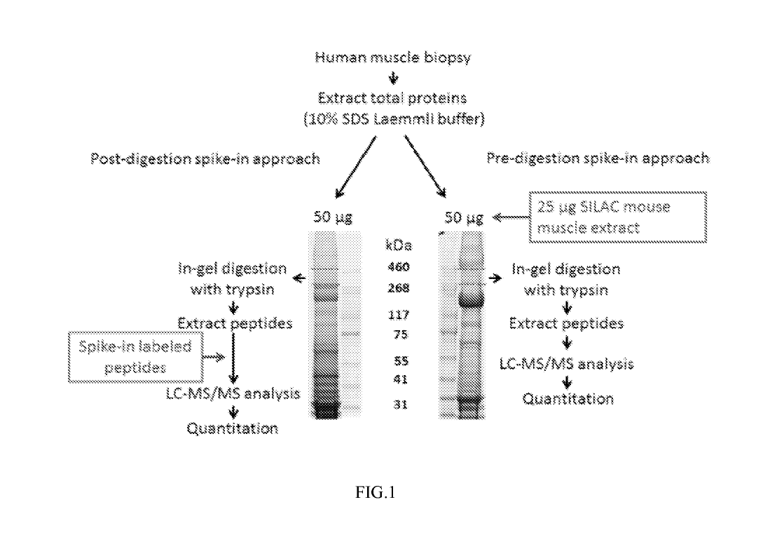

Two approaches were evaluated to quantitate dystrophin in skeletal muscle extracts (FIG. 1). In the first approach, called post-digestion spike-in approach hereafter, aliquots of 50 μg total protein from each muscle extract was diluted in LDS buffer (Invitrogen, Carlsbad, Calif.) and further fractionated by SDS-PAGE on a 3-8% Tris-Acetate midi gel (Invitrogen) for 1 hour at 160 V. Gels were stained with BioSafe commassie (BioRad) for 1 hour. The upper section of the gel (300 to 450 kDa) containing dystrophin protein and other skeletal muscle proteins was excised and processed for in-gel digestion as previously described [11] using proteomics grade trypsin (Promega, Madison, Wis.). Peptides were extracted from the gel and spiked with known amounts (14 nM, 40 nM, 80 nM and 160 nM) of custom synthesized stable isotope labeled dystrophin peptides as well as an internal control filamin c peptide. In the second approach, called pre-digestion spike-in approach hereafter, aliquots of 50 μg of total protein from each muscle extract were spiked with 25 μg of muscle extract prepared from 13C6-Lysine labeled SILAC mouse. The mixture was then fractionated by SDS-PAGE on a 3-8% Tris-Acetate gel and gel bands in the 300-450 kDa were processed as above. To determine the limit of detection (LOD) and limit of quantitation (LOQ) of dystrophin protein in a complex muscle extract we used a muscle extract from a DMD patient, completely lacking dystrophin protein, and mixed at different ratios with a muscle extract from a healthy donor that expresses dystrophin as follows: DMD to normal 100%/0%, 95%/5%, 90%/10%, 75%/25%, 50%/50%, 25%/75%, 10%/90%, 5%/95%, 0%/100%. The final protein content in each mixture was kept constant at 50 μg. Each sample was then spiked with 25 μg of muscle extract prepared from 13C6-Lys labeled SILAM mouse to quantitate human dystrophin. Samples were analyzed using two different types of mass spectrometry instruments. For analysis by LTQ-Orbitrap-XL (Thermo), concentrated peptides from each spiked sample above were dissolved in 10 μL of 0.1% TFA solution and 6 μL was injected via an autosampler onto a Symmetry C18 trap column (5 μm, 300 μm i.d., 23 mm, Waters) for 10 min at a flow rate of 10 μm/min, 100% A. The sample was subsequently separated by a C18 reverse-phase column (3 μm, 200A, 100 μmx×15 cm, Magic C18, Michrom

Bioresources) at a flow rate of 330 nL/min using an Eksigent nano-hplc system (Dublin, Calif.). The mobile phases consisted of water with 0.1% formic acid (A) and 90% acetonitrile (B). A 65 minute linear gradient from 5 to 40% B was employed. Eluted peptides were introduced into the mass spectrometer via a Michrom Bioresources CaptiveSpray. The spray voltage was set at 1.4 kV and the heated capillary at 200° C. The LTQ-Orbitrap-XL was operated in data-dependent mode with dynamic exclusion in which one cycle of experiments consisted of a full-MS in the Orbitrap (300-2000 m/z) survey scan in profile mode, resolution 30,000, and five subsequent MS/MS scans in the LTQ of the most intense peaks in centroid mode using collision-induced dissociation with the collision gas (helium) and normalized collision energy value set at 35%.

For Q Exactive analysis, concentrated peptides from each spiked sample above were redissoleved in 20 μL of 90:10 water:acetonitrile with 0.1% TFA containing 2.5 fmol/μL of trainer peptide mixture. Sample were then injected via autosampler followed by separation on a

HALO C18 column (75 μm ID, 10 cm, 2.7 μm) with a gradient of 0 to 35% (A: 0.1% formic acid water, B: 0.1% formic acid acetonitrile) at a flow rate of 400 nL/min on a Thermo Scientific Easy 1000 connected to a nanoESl flex source. For the peptide coverage discovery experiment, the Q Exactive was operated with a full scan (380-1500 m/z) at 70,000 resolution followed by MS/MS of the top ten most abundant ions at 17,500 resolution. For quantitative acquisition, the Q Exactive was operated with a full scan (380-1500 m/z) at 35,000 resolution followed by timed targeted MS/MS of dystrophin and internal stable isotope labeled standard peptides at 35,000 resolution.

### Protein Identification and Quantitation

LTQ-Orbitrap XL Raw files were searched using Sequest in the Bioworks Browser (Thermo) software against a tryptic human database with 2 missed cleavages, 50 ppm mass accuracy, 1.5 Da fragment mass tolerance. Spiked peptides were identified and elution times documented. Dystrophin was quantitated by comparing the unlabeled to the spiked-in heavy isotope profile for each peptide. Q Exactive Raw files were searched with Proteome Discoverer 1.3 (ThermoFisher Scientific) using SEQUEST and the human refseq. Database (release 47, ftp://ftp.ncbi.nih.gov/refseq/H_sapiens/mRNA_Prot/, with 34,340 entries). Search parameters allowed trypsin to cleave after Lysine and Arginine with 2 missed cleavages. Precursor ion mass tolerance was set to 10 ppm and fragment ion mass tolerance was 20 ppm. The dynamic modification of methionine (oxidation=15.995 Da) and lysine (13C6=6.020 Da), was accepted on up to 4 residues per peptide. Within the Proteome Discoverer Software, SILAC pairs were identified using the “2 plex” workflow node, and all peptides were rescored using the Percolator algorithm node. Finally, peptides were filtered at 1% FDR. SILAC quantification from 25% and 75% normal were compared and proteins showing a consistent ratio were considered as controls. Peak areas and area ratios were calculated with Pinpoint software using recreated ion chromatograms at 25 ppm from the full scan MS/MS. The ratios of the integrated areas for endogenous peptides and [13C6 lys] labeled peptides were calculated to obtain peptide measures using multiple y-ion fragments per peptide. The average of these peptide ratios determined the relative amount of each protein.

High SDS Containing Buffer Enabled Efficient Dystrophin Extraction from Skeletal Muscle Biopsies.

Dystrophin protein is a low abundant protein, accounting for only 0.002% of total muscle protein. Since no amplification technologies exist for protein detection by mass spectrometry, efficient dystrophin extraction was essential. In addition to efficient extraction, enrichment of dystrophin protein from other more abundant muscle proteins prior to mass spectrometry was required in order to reduce the sample dynamic range and thereby ensure consistent detection of dystrophin. We evaluated a mild (RIPA buffer) and strong (10% SDS) buffer for muscle protein extraction. As expected the 10% SDS modified Laemmli buffer extracted approximately 10 times more total protein than the RIPA buffer from the same amount of muscle tissue. Indeed, about 8 μg and 54 μg of total proteins were extracted from 1 mg of fresh muscle tissue using RIPA and 10% SDS modified Laemmli buffer, respectively. The increased extraction efficiency of the SDS buffer also increased the dynamic range of the sample by extracting more of the highly abundant skeletal muscle proteins such as myosins, titin and nebulin. To overcome the dynamic range limitations, total muscle extract from each of the RIPA and SDS preparation was further separated on a 3-8% Tris-Acetate gel. The region on the gel corresponding to the migration zone of dystrophin and/or truncated Becker-like dystrophin (300-450 kDa) was excised, and processed for mass spectrometry analysis as described above. Dystrophin protein was identified by 19 and 50 peptides when muscle sample was extracted with RIPA and 10% SDS modified Laemmli buffers, respectively (Table 1).

TABLE 1

| | # of missed | |
|---|---|---|
| Peptide | cleavages | SEQ ID NO: |
| IFLTEQPLEGLEK | 0 | 6441 |
| QLGEVASFGGSNIEPSVR | 0 | 6442 |
| VLSQIDVAQK | 0 | 6443 |
| TLNATGEEIIQQSSK | 0 | 6444 |
| VLSQIDVAQK | 0 | 6445 |
| SLEGSDDAVLLQR | 0 | 6446 |
| LLEQSIQSAQETEK | 0 | 6447 |
| QLNETGGPVLVSAPISPEEQDKLENK | 1 | 6448 |
| LQELQEATDELDLK | 0 | 6449 |
| VLQEDLEQEQVR | 0 | 6450 |
| TKEPVIMSTLETVR | 1 | 6451 |
| ASIPLKELEQFNSDIQK | 1 | 6452 |
| LLVEELPLR | 0 | 6453 |
| VLM*DLQNQK | 0 | 6454 |
| VM*VGDLEDINEMIIK | 0 | 6455 |
| LGLLLHDSIQIPR | 0 | 6456 |
| VAAAETAKHQAK | 1 | 6457 |
| LLQVAVEDRVR | 1 | 6458 |
| IFLTEQPLEGLEKLYQEPR | 1 | 6459 |
| HAQEELPPPPPQK | 0 | 6460 |
| VLSQIDVAQKK | 1 | 6461 |
| LLRDYSADDTR | 1 | 6462 |
| QAPIGGDFPAVQK | 0 | 6463 |
| RPQLEELITAAQNLK | 0 | 6464 |
| VHALNNVNK | 0 | 6465 |
| VM*VGDLEDINEM*IIKQK | 1 | 6466 |
| QITVDSEIRK | 1 | 6467 |
| SDSSQPM*LLR | 0 | 6468 |
| QAEGLSEDGAAM*AVEPTQIQLSKR | 1 | 6469 |
| LLDLLEGLTGQKLPK | 1 | 6470 |
| QASEQLNSR | 0 | 6471 |
| ELPPEERAQNVTR | 1 | 6472 |
| LLDLLEGLTGQKLPKEK | 2 | 6473 |
| GDNLQQR | 0 | 6474 |
| KLQDASR | 1 | 6475 |
| SPAQILISLESEERGELER | 1 | 6476 |
| FAAISHR | 0 | 6477 |
| KLQDVSM*K | 1 | 6478 |
| LQDVSM*KFR | 1 | 6479 |
| KMEEEPLGPDLEDLKR | 2 | 6480 |
| DYSADDTR | 0 | 6481 |
| TIITDRIER | 1 | 6482 |
| HSEDNPNQIR | 0 | 6483 |
| IQSDLTSHEISLEEMKK | 1 | 6484 |
| LYQEPR | 0 | 6485 |
| ASIPLKELEQFNSDIQK | 1 | 6486 |
| LRQLLEQPQAEAK | 1 | 6487 |
| DYSADDTRK | 1 | 6488 |
| ELPPEER | 0 | 6489 |
| NKTSNQEAR | 1 | 6490 |
| ATM*QDLEQR | 0 | 6491 |
| DQAANLM*ANR | 0 | 6492 |
| TAALQSATPVER | 0 | 6493 |
| QLLEQPQAEAK | 0 | 6494 |
| EEAEQVLGQAR | 0 | 6495 |
| ELEQFNSDIQK | 0 | 6496 |
| ILADLEEENR | 0 | 6497 |
| VGNILQLGSK | 0 | 6498 |
| SAVEGM*PSNLDSEVAWGK | 0 | 6499 |
| LLVSDIQTIQPSLNSVNEGGQK | 0 | 6500 |

### Dystrophin Quantitation by Mass Spectrometry

Two approaches to quantitate dystrophin protein in total muscle protein extracts were evaluated(Figure 1): (i) Post-Digestion spike-in approach with stable isotope labeled custom synthesized standard dystrophin peptides; and (ii) Pre-Digestion spike-in approach with stable isotope labeled mouse muscle protein extract prior to gel separation and in-gel digestion.

### Post-Digestion Spike-In Approach

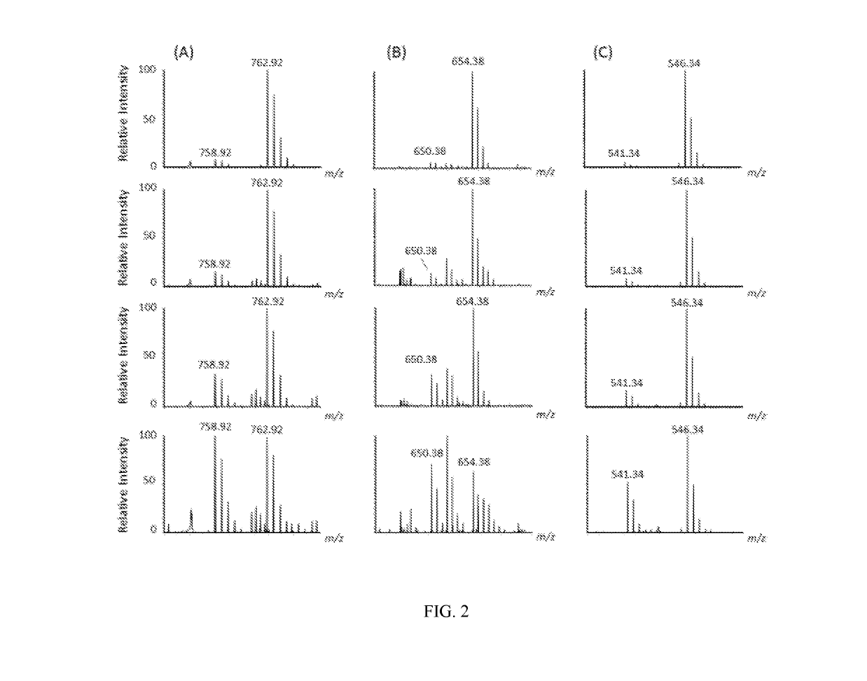

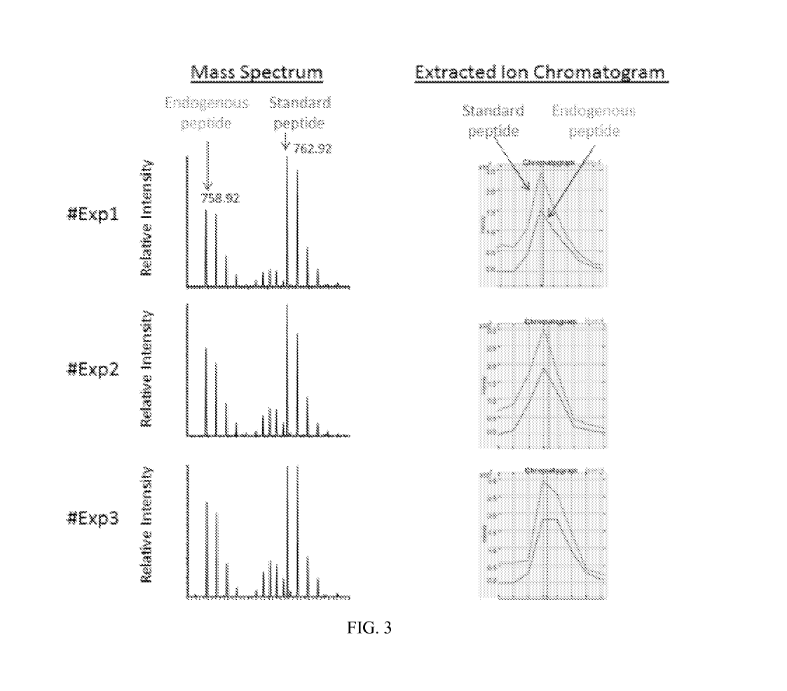

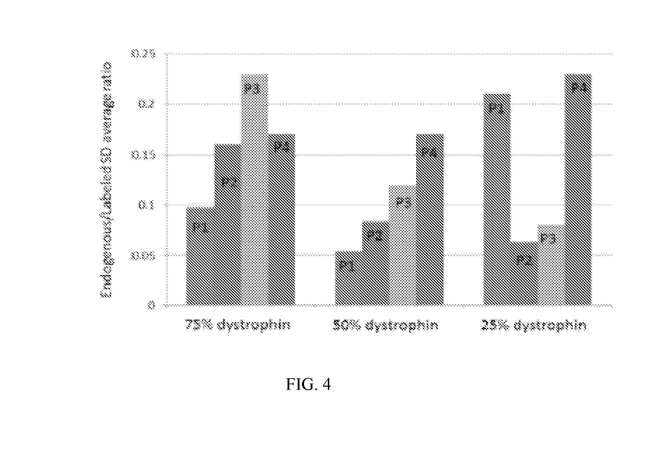

For the post-digestion spike-in approach, three stable isotope labeled dystrophin peptides were custom synthesized. The sequences of these peptides were chosen based on their consistent and reproducible detection by LC-MS/MS following in-gel digestion of SDSPAGE fractionated muscle extracts, different retention times, amino acid content (e.g. absence of highly modifiable residues such as cysteine and methionine), as well as the location within the dystrophin sequence (e.g. distributed throughout the N-terminal, middle and C-terminal domain). Additionally, a stable isotope labeled peptide for filamin c was also custom synthesized and used as an internal standard. The sequences of the standard peptides, their retention time, observed molecular masses as well as their location within the dystrophin sequence were determined. First the post-digestion spike-in approach was optimized for the amount of stable isotope labeled standard peptides to be used for spike-in. In-gel digested dystrophin containing bands were prepared from replicate SD S-PAGE fractionation of 50 μg of muscle protein extracts of a healthy donor and spiked with varying amounts of stable isotope labeled standard peptides (14 nM, 40 nM, 80 nM and 160 nM). FIG. 2 shows MS spectra of the unlabeled peptide from the endogenous dystrophin and its corresponding heavy stable isotope spiked-in standard at varying concentrations. From this preliminary study, the optimal amount of stable isotope labeled peptides to spike-in ranged between 14 and 40 nM. Spiking amounts below 14 nM resulted in poor signal to noise spectra while spiking amounts above 40 nM resulted in increased dynamic range between the standard peptide and the endogenous peptide ion intensities reducing the linear range for quantitation. To test the reproducibility of the post-digestion spike-in approach, triplicate extraction experiments performed on the same muscle biopsy obtained from a healthy donor were processed and spiked with an optimal amount of 30 nM of stable isotope labeled standard peptide mixture. FIG. 3 shows the reproducibility of the triplicate assay with a CV of less than 7%. Unfortunately, the reproducibility of the experiment did not hold across the three different dystrophin targeted peptides for a titration curve of endogenous dystrophin protein including the internal control protein filamin c (FIG. 4). This was attributed to the inherent variability of in-gel digestion. Therefore, the post-digestion spike-in approach suffered from high sample handling errors.

### Pre-Digestion Spike-In Approach

In order to overcome in-gel digestion variability a pre-digestion spike-in approach was evaluated using 13C6-Lysine fully labeled dystrophin protein obtained from muscle extracts prepared from a SILAC mouse. In this method the sample is spiked at the protein level prior to electrophoresis and in-gel digestion, thus minimizing variation due to sample handling and greatly improving reproducibility. Since production of a fully labeled human dystrophin protein using transfection technology and over-expression in a labeled culture system was challenging due to the low transfection rate of the largest human gene [12], use of 13C6-Lysine labeled dystrophin prepared from a SILAC mouse was employed [9,13]. Mouse dystrophin and human dystrophin have 91% sequence homology. Previously a fully 13C6-Lysine labeled SILAC mouse had been generated [8]. Skeletal muscle from the SILAC mouse was sectioned by cryostat, total protein containing 0.002% of fully labeled dystrophin were extracted using the 10% SDS modified Laemmli buffer then used as a spike-in standard in human muscle extracts. Spiked human muscle extracts were further fractionated by SDS-PAGE and processed for mass spectrometry analysis using the Q Exactive instrument as described above. In a discovery run, 43 unique dystrophin peptides were detected with good coverage throughout the entire dystrophin sequence. Of these 43 unique peptides, five terminated in lysine and had 100% sequence homology with mouse tryptic peptides and therefore were used to quantitate dystrophin in human muscle extracts. In addition to these dystrophin overlapping peptides between human and mouse, other lysine terminating peptides belonging to several other skeletal muscle proteins also had 100% sequence homology between human and mouse. This enabled the evaluation and selection of appropriate internal standards for accurate dystrophin quantitation.

### Limit of Detection and Limit of Quantitation of Dystrophin Protein in Total Muscle Extract

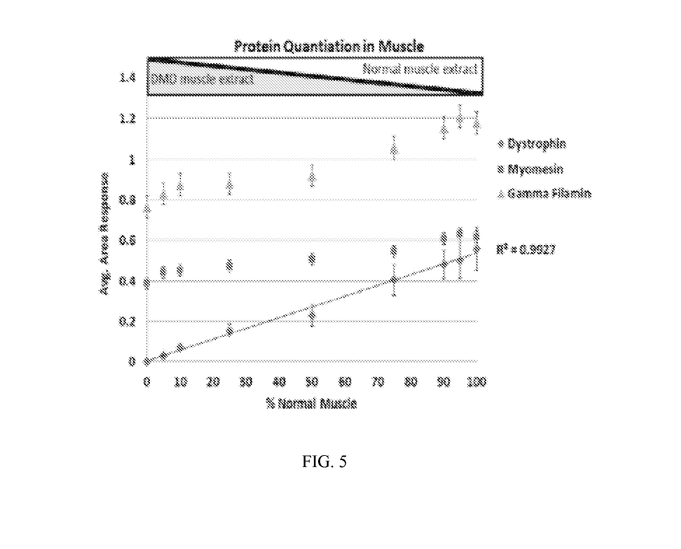

To evaluate the LOD and LOQ of the pre-digestion spike-in approach, targeted mass spectrometry analysis was used on the five dystrophin peptides detected above as well as a few internal control peptides from myomesin and filamin c. Dystrophin protein was targeted for quantitation in the following combination of normal and DMD muscle extracts: 0%/100%, 5%/95%, 10%/90%, 25/75%, 50/50%, 75/25%, 90/10%, 95/5%, 100/0%. Each mixture had a final protein content of 50 μg. The range of dystrophin protein expression, from 0% to 100% of normal, is thus artificially generated to represent possible amounts of dystrophin protein in Becker patients or in Duchenne patients receiving treatments aiming to restore dystrophin expression [3-7]. Each combination mixture above (50 μg final total protein) was then spiked with SILAC 13C6-Lysine labeled SILAC mouse muscle extract (25 μg). Samples were fractionated on a 3-8% Tris-Acetate gel, bands in the dystrophin protein region (300-450 kDa) were excised, in-gel digested and analyzed by LC-MS/MS on a Q Exactive mass spectrometer using a timed targeted MS/MS method. Unlabeled and labeled dystrophin, filmain c and myomesin peptides from both the human muscle extract and the spike-in SILAC mouse labeled extract were targeted for MS/MS analysis. Selected transition ions were used for quantitation in each sample. The average ratio of labeled to unlabeled peptide intensities for dystrophin, filamin c and myomesin were plotted against the different muscle combination mixtures resulting in a calibration curve with an R2 of 0.99 (FIG. 5). While the amount of dystrophin increased with increasing % of normal muscle extract in the mixtures, two muscle specific proteins, filamin c and myomesin, remained relatively unchanged. These two proteins are therefore ideal candidates as internal controls. Note that filamin c and myomesin did show a slight increase in abundance as the % of normal increased (FIG. 5), and this is expected since the amount of muscle present in dystrophic tissue is slightly decreased and is replaced by fibrotic tissue (e.g. hallmark of DMD pathogenesis) [14]. Therefore, monitoring of internal standards across the curve allows for improved accuracy and normalization.

### Absolute Quantitation of Dystrophin in Becker Muscular Dystrophy Patients

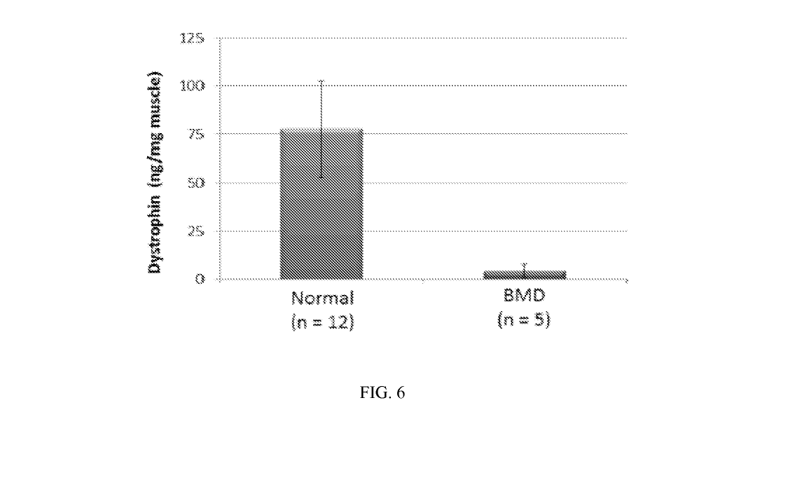

The absolute amount of dystrophin in muscle biopsies from normal donors (n=12) and Becker muscular dystrophy patient donors (n=5) was determined. Dystrophin in normal muscle ranged from 0.001 to 0.004% of total striated muscle, consistent with previous findings [2]. The Becker samples also showed a wide range of dystrophin expression at approximately 10% of the level found in normal muscle (FIG. 6). Dystrophin protein represents approximately 0.002% of total striated muscle protein and since the amount of spike-in standard is known, we estimated the total amount of dystrophin in healthy individuals to be 75±15 ng per mg of muscle while in Becker patients the amount can range from 0 to 7 ng per mg of muscle (FIG. 6).

### Extension of Mass Spectrometry SILAC Methods to Multiple Proteins.

The methods for quantitation of dystrophin, where alignments of peptides showing 100% homology between mouse SILAC skeletal muscle tissue proteins and human skeletal muscle tissue proteins, was extended to a survey of all paired mouse/human peptides (Tables 3-6). Muscle proteins were extracted using two different solubilization buffers (RIPA, SDS), fractionated on SDS-PAGE gels, mouse and human muscle samples mixed (as described above) and gel fragments subjected to trypsin digestion with detection of 100% conserved peptides on the Q-exactive mass spectrometer (Table 2; Table S2 [spreadsheet]).

TABLE 2

| | RIPA | RIPA | SDS | SDS |
|---|---|---|---|---|
| | Normal | DMD | Normal | DMD |
| Number of | 2,428 | 2,281 | 4,400 | 2,513 |
| paired | | | | |
| mouse/human | | | | |
| peptides | | | | |
| identified | | | | |

From this data generation, between 2,281 to 4,400 100% homologous mouse/human peptide pairs were identified, enabling accurate quantitation of 1,172 proteins from skeletal muscle (Table 7). By extension, similar data would be expected to be obtained from any other mixed human—mouse SILAC tissue pair, resulting in the ability to quantitate a wide range of human proteins from a wide range of clinical tissue samples.

Lengthy table referenced here
US20150168425A1-20150618-T00001
Please refer to the end of the specification for access instructions.

Lengthy table referenced here
US20150168425A1-20150618-T00002
Please refer to the end of the specification for access instructions.

Lengthy table referenced here
US20150168425A1-20150618-T00003
Please refer to the end of the specification for access instructions.

Lengthy table referenced here
US20150168425A1-20150618-T00004
Please refer to the end of the specification for access instructions.

Lengthy table referenced here
US20150168425A1-20150618-T00005
Please refer to the end of the specification for access instructions.

Extension of Mass Spectrometry Human Cell Culture (i.e. Human Muscle Cell Culture, Such as Myotube Cell Culture) Methods to Multiple Proteins.

The methods for quantitation of dystrophin, where alignments of peptides showing 100% homology between mouse SILAC skeletal muscle tissue proteins and human skeletal muscle tissue proteins, and the extension described above to a survey of all paired mouse/human peptides (Tables 3-6), may also be extended to methods using a labeled sample obtained from human cell culture, such as a human muscle cell culture, or a human myotube culture. Labeled standard made in human myotube culture may be used to survey a substantial number of proteins expressed in human myotube cells using methods similar to those previously described.

As shown in Table 8, about 3704 proteins expressed by myotubes may be surveyed, detected, and/or quantitated using methods similar to those described above. By extension, similar data would be expected to be obtained from any other labeled human cell line culture and corresponding biological sample of interest, resulting in the ability to quantitate a wide range of human proteins from a wide range of clinical tissue samples.

Lengthy table referenced here
US20150168425A1-20150618-T00006
Please refer to the end of the specification for access instructions.

### Modifications and Other Embodiments

Various modifications and variations of the described mouse/human paired peptide quantitation methods, and human cell culture/human quantitation methods, and methods of their use as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the medical, biological, molecular biological, chemical or pharmacological arts or related fields are intended to be within the scope of the following claims.

### Incorporation by Reference

Each document, patent, patent application or patent publication cited by or referred to in this disclosure is incorporated by reference in its entirety, especially with respect to the specific subject matter surrounding the citation of the reference in the text. However, no admission is made that any such reference constitutes background art and the right to challenge the accuracy and pertinence of the cited documents is reserved.

### REFERENCES

1. Atkinson, Arthur J., Wayne A. Colburn, Victor G. DeGruttola, David L. DeMets, Gregory J. Downing, Daniel F. Hoth, John A. Oates et al. “Biomarkers and surrogate endpoints: Preferred definitions and conceptual framework*.” Clinical Pharmacology & Therapeutics 69, no. 3 (2001): 89-95.

2. Hoffman E P, Brown R H Jr, Kunkel L M. Dystrophin: the protein product of the Duchenne muscular dystrophy locus. Cell. 1987; 51:919-928.

3. Sebahattin C, Virginia A, Michela G, Lucy F, Silvia T, et al. Exon skipping and dystrophin restoration in patients with Duchenne muscular dystrophy after systemic phosphorodiamidate morpholino oligomer treatment: an open-label, phase 2, dose-escalation study. Lancet. 2011; 378:595-605.

4. Finkel R S. Read-through strategies for suppression of nonsense mutations in Duchenne/Becker muscular dystrophy: aminoglycosides and ataluren (PTC124). J Child Neurol. 2010; 25:1158-1164.

5. Nelson S F, Crosbie R H, Miceli M C, Spencer M J. Emerging genetic therapies to treat Duchenne muscular dystrophy. Curr Opin Neurol. 2009; 22:532-538.

6. Hoffman E P, Bronson A, Levin A A, Takeda S, Yokota T, et al. Restoring dystrophin expression in duchenne muscular dystrophy muscle progress in exon skipping and stop codon read through. Am J Pathol. 2011; 179:12-22. [

7. Goemans N M, Tulinius M, van den Akker J T, Burm B E, Ekhart P F, et al. Systemic administration of PRO051 in Duchenne's muscular dystrophy. N Engl J Med. 2011; 364:1513-1522.

8. Sharma N, Medikayala S, Defour A, Rayavarapu S, Brown K J. Use of quantitative membrane proteomics identifies a novel role of mitochondria in healing injured muscles. J Biol Chem. 2012; 287:30455-30467.

9. Kruger M, Moser M, Ussar S, Thievessen I, Luber C A, et al. SILAC mouse for quantitative proteomics uncovers kindlin-3 as an essential factor for red blood cell function. Cell. 2008; 134:353-364.

10. Hoffman E P, Fischbeck K H, Brown R H, Johnson M, Medori R, et al. Characterization of dystrophin in musclebiopsy specimens from patients with Duchenne's or Becker's muscular dystrophy. N Engl J Med. 1988; 318:1363-1368.

11. Jensen O N, Wilm M, Shevchenko A, Mann M. Sample preparation methods for mass spectrometric peptide mapping directly from 2-DE gels. Methods Mol Biol. 1999; 112:513-530.

12. Tennyson C N, Klamut H J, Worton R G. The human dystrophin gene requires 16 hours to be transcribed and is cotranscriptionally spliced. Nat Genet. 1995; 9:184-190.

13. McClatchy, D B.; Yates, J R, III. Stable Isotope Labeling of Mammals (SILAM). 2008.

14. Ardite E, Perdiguero E, Vidal B, Gutarra S, Serrano A L, et al. PAI-1-regulated miR-21 defines a novel age-associated fibrogenic pathway in muscular dystrophy. J Cell Biol. 2012; 196:163-175.

15. Lederfein D, Levy Z, Augier N, Mornet D, Morris G et al., A 71-kilodalton protein is a major product of the Duchenne muscular dystrophy gene in brain and other nonmuscle tissues. Proc Natl Acad Sci. 1992; 89:5346-5350.

16. van Essen A J, Busch H F M, to Meerman G J, ten Kate L P. Birth and population prevalence of Duchenne muscular dystrophy in The Netherlands. Hum Genet 1992; 88:258-266.

17. Bushby K M D, Gardner-Medwin D, Nicholson L V B, Johnson M A, Haggerty I D et al., The clinical, genetic and dystrophin characteristics of Becker muscular dystrophy. J Neurol. 1993; 240:105-112.

18. Anthony K, Sebahattin C, Silvia T, Giorgio T, Lucy F et al., Dystrophin quantification and clinical correlations in Becker muscular dystrophy; implications for clinical trials. Brain. 2011; 134:3547-3559.

19. Yokota T, Lu Q L, Partridge T, Kobayashi M, Nakamura A et al., Efficacy of systemic morpholinon exon skipping in Duchenne dystrophy dogs. Ann Neurol. 2009; 65:667-676.

20. Lu Q L, Yokota T, Takeda S, Garcia L, Muntoni F et al. The status of exon skipping as a therapeutic approach to Duchenne muscular dystrophy. Mol Ther. 2011; 19:9-15.

LENGTHY TABLES
The patent application contains a lengthy table section. A copy of the table is available in electronic form from the USPTO web site (http://seqdata.uspto.gov/?pageRequest=docDetail&DocID=US20150168425A1). An electronic copy of the table will also be available from the USPTO upon request and payment of the fee set forth in 37 CFR 1.19(b)(3).

1. A method for identifying candidate peptide markers for a protein from a biological sample of interest, the method comprising the steps of:
extracting proteins from the biological sample of interest and a corresponding biological sample from an animal that has been labeled with one or more stable isotopic amino acids;
mixing a quantity of protein from the biological sample of interest with a quantity of protein from the corresponding biological sample from the labeled animal to form a spiked sample;
separating the spiked sample based on a physical property;
digesting the proteins of interest using a protease;
subjecting the digested samples to mass spectrometry analysis; and
identifying unique candidate peptide markers that correspond to a protein.

2. A method for quantifying expression of a biomarker from a biological sample obtained from a subject with a disease or disorder, the method comprising the steps of:
extracting proteins from the biological sample of interest;
mixing a known quantity of protein from the biological sample of interest with a known quantity of protein from a the corresponding biological sample from a labeled animal, a labeled human cell culture, or a labeled synthetic peptide corresponding to a protein of interest to form a spiked sample;
separating the spiked sample based on a physical property;
digesting the proteins of interest using a protease;
subjecting the digested samples to mass spectrometry analysis;
determining intensities of unique candidate peptide markers that correspond to a proteins of interest; and
quantifying the amount of the protein of interest in the sample based on the relative intensity of the unique candidate peptide markers.

3. The method of claim 2, wherein the step of quantifying comprises comparing peak areas and area ratios of one or more unique candidate peptide markers from the biological sample of interest to the peak areas and area ratios of one or more labeled peptide markers.

4. The method of claim 2, wherein the step of quantifying comprises comparing the relative intensities to a standard curve generated by creating a series of dilutions of a biological sample from a normal subject with a biological sample from a subject with a disease or disorder, wherein the amount of total protein in the mixed sample remains constant; mixing a known quantity of protein from the mixed sample with a known quantity of protein a corresponding biological sample from a labeled animal or a labeled synthetic peptide corresponding to a protein of interest to form a spiked sample; separating the spiked sample based on a physical property; digesting the proteins of interest using a protease; subjecting the digested samples to mass spectrometry analysis; and generating a titration curve based on the intensities obtained for each sample.

5. A method for quantifying dystrophin expression from a biological sample obtained from a subject with Duchenne's Muscular Dystrophy, the method comprising the steps of:
extracting proteins from the biological sample of interest;
mixing a known quantity of total protein from the biological sample of interest with a known quantity of total protein a the corresponding biological sample from a labeled animal, a human cell culture, or a labeled synthetic peptide corresponding to dystrophin to form a spiked sample;
separating the spiked sample based on a physical property;
digesting the proteins of interest using a protease;
subjecting the digested samples to mass spectrometry analysis;
quantifying the amount of the protein of interest in the sample based on the relative intensity of the unique candidate peptide markers.

6. The method of claim 5, wherein the step of quantifying comprises comparing peak areas and area ratios of one or more unique candidate peptide markers from the biological sample of interest to the peak areas and area ratios of one or more labeled peptide markers.

7. The method of claim 5, wherein the step of quantifying comprises comparing the relative intensities to a standard curve generated by creating a series of dilutions of a biological sample from a normal subject with a biological sample from a subject with a disease or disorder, wherein the amount of total protein in the mixed sample remains constant; mixing a known quantity of protein from the mixed sample with a known quantity of protein a corresponding biological sample from a labeled animal, a labeled cell culture, or a labeled synthetic peptide corresponding to a protein of interest to form a spiked sample; separating the spiked sample based on a physical property; digesting the proteins of interest using a protease; subjecting the digested samples to mass spectrometry analysis; and generating a titration curve based on the intensities obtained for each sample.

8. The method of claim 5, wherein the subject is being treated with a pharmaceutical composition.

9. The method of claim 5, wherein the one or more peptides are selected from Table 1.

10. The method of claim 9, wherein the one or more peptides are selected from QAPIGGDFPAVQK, VLSQIDVAQK, IFLTEQPLEGLEK, TLNATGEEIIQQSSK, and VHALNNVNK.

11. The method of claim 2, when the proteins are extracted using a buffer comprising 10% sodium-dodecyl sulfate (SDS).

12. The method of claim 2, wherein the known quantity of total protein corresponding to the biological sample from a labeled animal or a labeled human cell culture is half the known quantity of total protein from the biological sample of interest.

13. The method of claim 12, wherein the known quantity of total protein corresponding to the biological sample of interest is 50 μg and the known quantity of total protein the corresponding to the biological sample from a labeled animal or a labeled human cell culture is 25 μg.

14. The method of claim 2, wherein the protease is trypsin.

15. The method of claim 2, wherein the spiked sample is separated based on size, charge, shape or a combination thereof using electrophoresis.

16. The method of claim 15, wherein the spiked sample is separated using gel electrophoresis.

17. The method of claim 16, further comprising the step of excising a portion of the gel containing the proteins of interest prior to the digesting step.
